(19) United States
(12) Patent Application Publication
(10) Pub. No.: US 2026/0199809 A1
(43) Pub. Date: Jul. 16, 2026

(54) STREPTAVIDIN CHROMATOGRAPHIC MATERIALS AND METHODS OF USE THEREOF

(71) Applicant: Waters Technologies Corporation, Milford, MA (US)

(72) Inventors: Beatrice Muriithi, Attleboro, MA (US); Yeliz Tunc Sarisozen, Westford, MA (US); Nathan Canniff, Concord, MA (US); Martin Gilar, Franklin, MA (US); Kevin Wyndham, Worcester, MA (US)

(73) Assignee: Waters Technologies Corporation, Milford, MA (US)

(21) Appl. No.: 19/448,858

(22) Filed: Jan. 14, 2026

Related U.S. Application Data
(60) Provisional application No. 63/744,959, filed on Jan. 14, 2025.

(51) Int. Cl. B01D 15/38; B01D 15/22; B01J 20/26; B01J 20/28; B01J 20/289; B01J 20/32; C12N 15/10

(52) U.S. Cl. CPC B01D 15/3823; B01D 15/22; B01J 20/265; B01J 20/28004; B01J 20/289; B01J 20/321; B01J 20/3219; B01J 20/3268; C12N 15/101

(57) ABSTRACT

Disclosed herein are chromatographic materials comprising streptavidin molecules, wherein the streptavidin molecules are bound to biotin and one or more biotinylated affinity agents. The biotinylated affinity agent may be a biotinylated antibody, biotinylated antigen-binding fragment, or biotinylated oligonucleotide. The streptavidin chromatographic materials may be used to prepare affinity chromatographic materials. Due to biotin binding to the conjugated streptavidin, the materials provided herein have reduced to no streptavidin leachate, resulting in improved performance.

### CROSS-REFERENCE TO RELATED APPLICATION

This application claims priority from and the benefit of U.S. Application No. 63/744,959 filed on Jan. 14, 2025. The entire contents of this application are incorporated herein by reference.

### FIELD OF TECHNOLOGY

The present disclosure relates generally to methods of preparing streptavidin-conjugated chromatographic materials and methods of using said materials in affinity chromatography.

### BACKGROUND

Streptavidin is used in affinity chromatography applications due to its strong non-covalent bond with biotin and biotinylated molecules. Streptavidin is present as a tetramer with four binding pockets that bind to biotin (or a biotinylated molecule). The streptavidin tetramer is increasingly stabilized by binding of each of the four binding sites; however, in the absence of said binding, the streptavidin tetramer is susceptible to dissociation under harsh conditions such as high temperatures, strong denaturants, or low pH. This dissociation releases streptavidin monomers, streptavidin leachate, which can interfere with protein-protein interactions, data quality, and overall sample analysis. Accordingly, there exists a need in the art for methods of reducing streptavidin leachate from materials for affinity chromatography.

### SUMMARY OF THE TECHNOLOGY

The present technology provides streptavidin-conjugated chromatographic materials, such as particles, that result in reduced streptavidin leachate. The streptavidin-conjugated chromatographic material may include one or more biotinylated affinity agents and free biotin. By interacting the streptavidin-conjugated chromatographic material with both biotin and one or more biotinylated affinity agent, more binding sites on streptavidin may be occupied (e.g., by either the biotinylated affinity agent or the free biotin), thereby reducing streptavidin leachate.

Accordingly, in a first aspect, disclosed herein is a method of preparing an affinity chromatographic column, the method including contacting a chromatographic column with a solution including a mixture of a biotinylated affinity agent and free biotin. The chromatographic column includes a column body formed of a metal or a metal alloy, and the column body houses a plurality of streptavidin-conjugated nonporous particles.

In some embodiments, the biotinylated affinity agent and free biotin bind to accessible binding sites of the streptavidin molecules, such that at least 90% of accessible binding sites of the streptavidin molecules are bound with the biotinylated affinity agent or free biotin. In some embodiments, at least 95% of accessible binding sites of the streptavidin molecules are bound with the biotinylated affinity agent or free biotin. In some embodiments, the biotinylated affinity agent and free biotin are in a 1:1 molar ratio, 1:3 molar ratio, 1:5 molar ratio, 1:8 molar ratio, 1:10 molar ratio, 1:15 molar ratio, 1:20 molar ratio, 1:25 molar ratio, 1:30 molar ratio, 1:35 molar ratio, 1:40 molar ratio, 1:45 molar ratio, or 1:50 molar ratio. In some embodiments, the method is performed in less than 20 minutes.

In some embodiments, each particle of the plurality of streptavidin-conjugated nonporous particles includes: a nonporous polymer core; and a hydrophilic surface on an outer layer of the nonporous polymer core, wherein one or more streptavidin molecules are conjugated to the hydrophilic surface. The average size of the streptavidin-conjugated nonporous particle is from 1.0 μm to 10 μm.

In some embodiment, the nonporous polymer core has a gradient composition. In some embodiments, the nonporous polymer core includes divinylbenzene monomers and styrene monomers. In some embodiments, the hydrophilic surface is selected from the group consisting of: (3-glycidyloxypropyl)trimethoxysilane, (3-glycidyloxypropyl)triethoxysilane, polyacrylate, glycidol, glyceroltriglycidyl ether, and poly(methyl acrylate).

In some embodiments, the one or more streptavidin molecules are conjugated to the hydrophilic surface of the particle via an epoxy linker. In some embodiments, the epoxy linker has a formula of

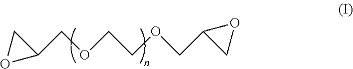

wherein n is between 1-12. In some embodiments, n is 1, 4, or 9. In some embodiments, n is 1.

In some embodiments, the plurality of streptavidin molecules conjugated to the hydrophilic surface provides a surface coverage of 2-6 μg/mg particle. In some embodiments, the biotinylated affinity agent is a biotinylated antibody or biotinylated antigen-binding fragment thereof, or a biotinylated oligonucleotide.

In a second aspect, disclosed herein is an affinity chromatographic column generated by the method of the first aspect. In some embodiments, at least a portion of an interior surface of the column body is coated with an alkylsilyl material. In some embodiments, the chromatographic column further includes frits within the column body, wherein the frits are coated with the alkylsilyl material. In some embodiments, the alkylsilyl material is a hydrophilic, non-ionic layer of polyethylene glycol silane.

In some embodiments, the column is characterized by a reduction in detectable leachate of streptavidin as determined by UV absorbance. In some embodiments, the column is characterized by a leachate absorbance value of <10 mAU as measured by UV absorbance at 280 nm. In some embodiments, the column is characterized by at least an 85% reduction in leachate absorbance as compared to a column that does not include streptavidin molecules that are bound with a mixture of the biotinylated affinity agent and the free biotin. In some embodiments, the leachate absorbance is determined at the 4^{th} peak eluted from the column as measured by UV absorbance at 280 nm. In some embodiments, the column has at least a 90% reduction or at least a 95% reduction in leachate absorbance. In some embodiments, the column is characterized by no detectable leachate of streptavidin as determined by UV absorbance.

In a third aspect, disclosed herein is an affinity chromatographic column including a column body formed of a metal or a metal alloy, the column body housing a plurality of streptavidin-conjugated nonporous particles, wherein at least 90% of accessible binding sites of the streptavidin molecules are bound with a biotinylated affinity agent or molecular biotin.

In some embodiments, each particle of the plurality of streptavidin-conjugated nonporous particles includes: a nonporous polymer core; a hydrophilic surface on an outer layer of the nonporous polymer core, wherein one or more streptavidin molecules are conjugated to the hydrophilic surface; and the average particle size of the streptavidin nanoparticle is from about 1.0 μm to about 10 μm.

In some embodiments, the affinity chromatography column includes the biotinylated agent and molecular biotin. In some embodiments, the affinity chromatography column includes the biotinylated agent and molecular biotin in a 1:1 molar ratio, 1:3 molar ratio, 1:5 molar ratio, 1:8 molar ratio, 1:10 molar ratio, 1:15 molar ratio, 1:20 molar ratio, 1:25 molar ratio, 1:30 molar ratio, 1:35 molar ratio, 1:40 molar ratio, 1:45 molar ratio, or 1:50 molar ratio.

In some embodiment, the nonporous polymer core has a gradient composition. In some embodiments, the nonporous polymer core includes divinylbenzene monomers and styrene monomers. In some embodiments, the hydrophilic surface is selected from the group consisting of: (3-glycidyloxypropyl)trimethoxysilane, (3-glycidyloxypropyl)triethoxysilane, polyacrylate, glycidol, glyceroltriglycidyl ether, and poly(methyl acrylate).

In some embodiments, the one or more streptavidin molecules are conjugated to the hydrophilic surface of the particle via an epoxy linker. In some embodiments, the epoxy linker has a formula of

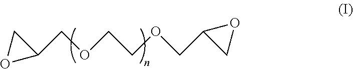

wherein n is between 1-12. In some embodiments, n is 1, 4, or 9. In some embodiments, n is 1.

In some embodiments, the plurality of streptavidin molecules conjugated to the hydrophilic surface provides a surface coverage of 2-6 μg/mg particle. In some embodiments, the biotinylated affinity agent is a biotinylated antibody or biotinylated antigen-binding fragment thereof, or a biotinylated oligonucleotide.

In some embodiments, the column is characterized by a reduction in detectable leachate of streptavidin as determined by UV absorbance. In some embodiments, the column is characterized by a leachate absorbance value of <10 mAU as measured by UV absorbance at 280 nm. In some embodiments, the column is characterized by at least an 85% reduction in leachate absorbance as compared to a column that does not include streptavidin molecules that are bound with a mixture of the biotinylated affinity agent and the free biotin. In some embodiments, the leachate absorbance is determined at the 4^{th} peak eluted from the column as measured by UV absorbance at 280 nm. In some embodiments, the column has at least a 90% reduction or at least a 95% reduction in leachate absorbance. In some embodiments, the column is characterized by no detectable leachate of streptavidin as determined by UV absorbance.

In some embodiments, the affinity chromatographic column of any one of claims 25-40, wherein the biotinylated affinity agent is a biotinylated antibody or biotinylated antigen-binding fragment thereof, or a biotinylated oligonucleotide.

In a fourth aspect, disclosed herein is a method of enriching a target analyte, the method including: i) providing the affinity chromatographic column of the second aspect, the affinity chromatographic column of the third column, or an affinity chromatographic column prepared according to the first aspect; ii) washing the affinity chromatographic column with a wash buffer; iii) applying a solution containing the target analyte to the affinity chromatographic column; and iv) washing the affinity chromatographic column with an elution buffer such that the target analyte is eluted from the column.

In some embodiments, the method further includes monitoring the eluent during step ii) with a UV detector. In some embodiments, there is a reduction in detectable leachate of streptavidin as determined by UV absorbance during step ii). In some embodiments, the UV absorbance at 280 nm is <10 mAU during step ii). In some embodiments, there is at least an 85% reduction in leachate absorbance as compared to a column that does not include streptavidin molecules that are bound with a mixture of the biotinylated affinity agent and the free biotin. In some embodiments, the leachate absorbance is determined at the 4^{th} peak eluted from the column as measured by UV absorbance at 280 nm. In some embodiments, the column has at least a 90% reduction or at least a 95% reduction in leachate absorbance. In some embodiments, there is no detectable leachate of streptavidin as determined by UV absorbance during step ii).

### BRIEF DESCRIPTION OF THE DRAWINGS

The technology will be more fully understood from the following detailed description taken in conjunction with the accompanying drawings, in which:

FIG. 1 shows the UV absorbance at 260 nm of the of the eluant obtained from running a mobile phase of 100 mM of phosphate buffered saline (PBS; pH 7.4) at a flow rate of 0.15 mL/min. D-biotin and biotinylated dt₂₅ were added with 2 μL injections. Saturation (indicated by increase in absorbance at 260 nm) was observed after 30 injections.

FIG. 2 shows the UV absorbance of mRNA eluted from a biotin endcapped streptavidin-conjugated particle column. Four columns were prepared with (1) 28.5 nmol of dT₂₅ and no biotin; (2) 11.4 nmol of dT₂₅ and a 1:3 molar ratio of biotin; (3) 4.56 nmol of dT₂₅ and a 1:8 molar ratio of biotin; and (4) 0.97 nmol of dT₂₅ and a 1:40 molar ratio of biotin. The arrow in each trace shows the injection where increased mRNA in the eluent, as indicated by an increased UV absorbance.

### DETAILED DESCRIPTION

Disclosed herein are streptavidin-conjugated particles including a biotinylated affinity agent and molecular biotin, chromatographic materials and affinity chromatographic columns including such particles, methods for preparing such columns, and methods of use thereof. In order that the technology may be more readily understood, certain terms are first defined. In addition, it should be noted that whenever a value or range of values of a parameter are recited, it is intended that values and ranges intermediate to the recited values are also part of this disclosure. The word “about” if not otherwise defined means±5%. It is also to be noted that as used herein and in the claims, the singular forms “a,” “an” and “the” include plural references unless the context clearly dictates otherwise.

### Definitions

As used herein, the term “antibody” refers to an immunoglobin molecule that specifically binds to, or is immunologically reactive with, a particular antigen. This includes polyclonal, monoclonal, genetically engineering, and otherwise modified forms of antibodies, including but not limited to chimeric antibodies, camelids, monobodies, humanized antibodies, heteroconjugate antibodies (e.g., bi-, tri-, and quad-specific antibodies, diabodies), and antigen-binding fragments of antibodies, including, for example, Fab′, F(ab′)2, Fab, Fv, and scFv fragments. Unless otherwise indicated, the term “monoclonal antibody” is meant to include both intact molecules as well as antibody fragments that are capable of specifically binding to a target protein. As used herein, the Fab and F(ab′)2 fragments refer to antibody fragments that lack the Fc portion of an intact antibody.

As used herein, the term “antigen-binding fragment” refers to one or more fragments of an antibody that retain the ability to specifically bind to a target antigen. The antigen-binding function of an antibody can be performed by fragments of a full-length antibody. The antibody fragments can be, for example, a Fab, F(ab′)2, scFv, a camelid, an affibody, a nanobody, an aptamer, or a domain antibody.

As used herein, the term “bispecific antibody” refers to an antibody that is capable of binding at least two different antigens.

As used herein, the term “biotin” (also referred to as “vitamin B₇”, and “vitamin H”) refer to the molecule

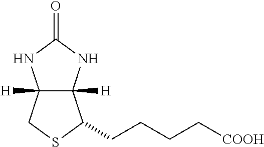 [SMILES: O=C(O)CCCC[C@@H]1SC[C@@H]2NC(=O)N[C@@H]21]

A “biotinylated species” (e.g., a biotinylated affinity agent) refers to a complex formed by covalently bonding a molecule of biotin to a second species (e.g., an affinity agent). “Free biotin”, as used herein, refers to a molecule of biotin which is not covalently attached to a second species (e.g., an affinity agent). Both biotin and biotinylated species may be conjugated (e.g., through a non-covalent interaction, e.g., a hydrogen bond) to another species (e.g., streptavidin).

As used herein, the term “biotinylated affinity agent” refers to a biotinylated molecule that can specifically bind to a target antigen or complementary nucleic acid sequence. The biotinylated affinity agent may be a biotinylated antibody or antigen-binding fragment thereof or a biotinylated oligonucleotide. The preparation of biotinylated molecules is a process well known and understood in the art. In some embodiments, the molecule is biotinylated with a biotin derivative, including but not limited to iminobiotin, desthiobiotin, disulfide biotin azide, disulfide biotin alkyne or other biotin derivatives.

As used herein, the term “biotin endcapped streptavidin-conjugated particles,” “biotin endcapped streptavidin-conjugated monoliths,” or “biotin endcapped streptavidin-conjugated membranes” refers to a particle, monolith, or membrane conjugated to one or more streptavidin molecules that are each associated with one or more biotinylated affinity agents and a biotin molecule. The streptavidin of a biotin endcapped streptavidin-conjugated particle may include fewer available binding sites than a streptavidin-conjugated particle which is not biotin endcapped.

As used herein, the term “nonporous” or “nonporous core” as used herein, refers to a material or a material region (e.g., the core) that has a pore volume that is less than 0.1 cc/g. Preferably, nonporous polymer cores have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA).

As used herein, the term “monolith” refers to a collection of individual particles packed into a bed formation, in which the shape and morphology of the individual particles are maintained. The particles are advantageously packed using a material that binds the particles together. Examples of suitable monoliths and binding materials are known in the art and further described in US Publication No. US 2023/0294073, incorporated herein by reference.

As used herein, the term “membrane” refers to a selective barrier, such as a semi-permeable barrier. A membrane may be a filtration membrane.

As used herein, the term “polyclonal antibody” refers to an antibody or a population of antibodies that has specificity to one or more antigens (such as, e.g., host cell proteins from a host cell line). A population of polyclonal antibodies recognize one or more distinct epitopes of the one or more antigens.

As used herein, the term “conjugate” refers to a compound formed by the chemical bonding of a reactive functional group of one molecule or macromolecule, such as streptavidin, with an appropriately reactive functional group of another molecule, such as an epoxide. An example of suitably reactive functional groups is a nucleophile/electrophile pair. For instance, the nucleophile may be an amine group from an amino acid of streptavidin, and the electrophile is an epoxide.

As used herein, the term “conjugated” refers to the linkage of two molecules formed by the chemical bonding of a reactive functional group of one molecule, such as streptavidin, with an appropriately reactive functional group of another molecule, such as an epoxide.

As used herein, the term “streptavidin leachate” refers to the dissociation of one or more streptavidin tetramers into streptavidin monomers, such that the monomers are not conjugated to a solid support, such as a nonporous particle described herein. Streptavidin leachate may be detected using known detection methods, including UV absorbance.

### Biotin Endcapped Streptavidin-Conjugated Particles

A biotin endcapped streptavidin-conjugated particle described herein may include a particle, streptavidin-conjugated to the surface of the particle, free biotin, and a biotinylated affinity agent. The free biotin and the biotinylated affinity agent may interact with binding sites on streptavidin, thereby preventing dissociation of the streptavidin tetramer and leeching of the streptavidin.

### Streptavidin-Conjugated Particles

In some embodiments, the particle may be of any size, for example from about 1 μm to about 50 μm (e.g., from about 1 μm to about 45 μm, from about 1 μm to about 40 μm, from about 1 μm to about 35 μm, from about 1 μm to about 30 μm, from about 1 μm to about 25 μm, from about 1 μm to about 20 μm, from about 1 μm to about 15 μm, from about 1 μm to about 14 μm, from about 1 μm to about 13 μm, from about 1 μm to about 12 μm, from about 1 μm to about 11 μm, from about 1 μm to about 10 μm, etc.). In some embodiments, the average particle in a plurality of particles may be a size of from about 1 μm to about 50 μm (e.g., from about 1 μm to about 45 μm, from about 1 μm to about 40 μm, from about 1 μm to about 35 μm, from about 1 μm to about 30 μm, from about 1 μm to about 25 μm, from about 1 μm to about 20 μm, from about 1 μm to about 15 μm, from about 1 μm to about 14 μm, from about 1 μm to about 13 μm, from about 1 μm to about 12 μm, from about 1 μm to about 11 μm, from about 1 μm to about 10 μm, etc.). In some embodiments, the average particle in a plurality of particles may be a size of from about 1 μm to about 10 μm.

In some embodiments, the particle includes a nonporous polymer core. In some embodiments, the nonporous particle core includes a polymer core. In some embodiments, the nonporous polymer core has a gradient composition. In some embodiments, the nonporous polymer core includes divinylbenzene monomers and styrene monomers and styrene monomers. In some embodiments, the particle polymer core was produced from divinylbenzene 80% (i.e., a mixture which is 80% divinylbenzene by weight and 20% non-divinylbenzene by weight. Some or all of the non-divinylbenzene material may be a stabilizer which prevents radical polymerization).

In some embodiments, the surface of the particle is a hydrophilic surface. In some embodiments, the surface of the particle is conjugated to one or more streptavidin via a linker moiety. In some embodiments, the linker moiety is an epoxy linker. In some embodiments, the epoxy linker is of formula (I)

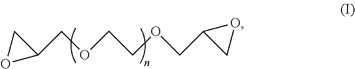

wherein n is any integer from 1 to 150. In some embodiments, n is from 1 to 12 (e.g., 1, 2, 3, 4, 5, 6, 7, 8, 9, 10, 11, or 12). In some embodiments, n is 1, 4, or 9. In some embodiments, n is 1.

### Biotinylated Affinity Agents

A streptavidin-conjugated particle described herein may be conjugated to one or more biotinylated affinity agent. A biotinylated affinity agent includes a biotin moiety bound to a molecule or macromolecule configured to bind a specific substrate (e.g., an antigen or antibody). The biotin moiety allows the biotinylated affinity agent to interact with the surface of the streptavidin-conjugated particle, associating the biotinylated affinity agent to the particle. Association of a biotinylated affinity agent to streptavidin may reduce the degradation of streptavidin (e.g., by reducing dissociation of a monomer from the streptavidin tetramer), thereby reducing streptavidin leachate from the particle.

A biotinylated affinity agent may be a biotinylated antibody, or a biotinylated antigen-binding fragment thereof. In some embodiments, the biotinylated affinity agent is a biotinylated oligonucleotide.

In some embodiments, the biotin moiety may be a biotin derivative. Exemplary biotin derivatives include iminobiotin, desthiobiotin, disulfide biotin azide, disulfide biotin alkyne or other biotin derivatives. Affinity agents may be biotinylated by any means known in the art.

In some embodiments, the surface coverage of streptavidin on the particle is from about 0.1 μg of streptavidin per mg of particle (i.e., about 0.1 μg/mg) to about 50 μg/mg (e.g., from about 0.2 μg/mg to about 45 μg/mg, from about 0.3 μg/mg to about 40 μg/mg, from about 0.4 μg/mg to about 35 μg/mg, from about 0.5 μg/mg to about 30 μg/mg, from about 0.6 μg/mg to about 25 μg/mg, from about 0.7 μg/mg to about 20 μg/mg, from about 0.8 μg/mg to about 15 μg/mg, from about 0.9 μg/mg to about 10 μg/mg, from about 1 μg/mg to about 8 μg/mg, from about 2 μg/mg to about 6 μg/mg, etc.).

### Mixtures of Biotin and Biotinylated Affinity Agents

Due to the size of the biotinylated affinity agent, one or more streptavidin binding sites on the streptavidin-conjugated particle may not be conjugated to the affinity agent. This reduces the stability of the streptavidin, which may result in an increased streptavidin leachate. The particles described herein stabilize streptavidin by binding free biotin to open streptavidin binding sites.

In some embodiments, the ratio of biotinylated affinity agent bound to streptavidin binding sites to free biotin bound to streptavidin binding sites is about 1:1, 1:3, 1:5, 1:8, 1:10, 1:15, 1:20, 1:25, 1:30, 1:35, 1:40, 1:45, or 1:50. In some embodiments, at least 1% of accessible binding sites of the streptavidin molecules are bound with the biotinylated affinity agent or free biotin (e.g., at least 2%, at least 3%, at least 4%, at least 5%, at least 6%, at least 7%, at least 8%, at least 9%, at least 10%, at least 11%, at least 12%, at least 13%, at least 14%, at least 15%, at least 20% at least 30%, at least 40%, at least 50%, at least 60%, at least 70%, at least 80%, at least 85%, at least 90%, at least 95%, at least 99%, etc.).

### Methods of Synthesis

Provided herein are methods for preparing biotin endcapped streptavidin-conjugated materials, such as particles. Biotin endcapped streptavidin-conjugated particles may be formed by first forming a particle (e.g., a particle described herein), reacting the particle with a linker (e.g., an epoxy linker) to form a particle with a functionalized surface, conjugating streptavidin to the surface of the particle, then binding a biotinylated affinity agent and free biotin to the streptavidin on the surface of the particle, thereby creating a biotin endcapped streptavidin-conjugated material.

In some embodiments, the surface of the particle includes a hydrophilic surface. In some embodiments, the hydrophilic surface layer is added after formation of the particle. In some embodiments, the hydrophilic surface layer is native to the particle.

### Methods of Forming Particles

An exemplary method for forming a particle is as follows. First, 561.1 g of reagent alcohol (90% ethanol, ^{~}5% methanol and ^{~}5% isopropanol), 16.9 g of polyvinylpyrrolidone (PVP-40, average molecular weight 40,000), 1.6 g of 2,2′-Azobis(2-methylpropionitrile) (AIBN), 6.7 g of Triton™ N-57, 80.1 g of styrene and 2.4 g of poly(propylene glycol) dimethacrylate (average molecular weight 560) were charged into a reactor. After purging with nitrogen, the reaction mixture was heated to 70° C. with stirring and was held at 70° C. until the completion of all the reaction steps. Then, the reaction mixture was held at 70° C. for 3 hours, a solution containing 52.0 g of divinylbenzene (80% by weight), 24.0 g of styrene, 51.0 g of PVP-40, 1080.4 g of reagent alcohol (90% ethanol, ^{~}5% methanol and ^{~}5% isopropanol) and 54.1 g of p-xylene was added to the reaction mixture at a constant flow rate over two hours. Finally, after the completion of solution charge in step two, a primer coating solution containing 31.2 g of glycidyl methacrylate (GMA), 6.2 g of ethylene glycol dimethacrylate (EDMA), 12.9 g of PVP-40 and 381.9 g of reagent alcohol (90% ethanol, ^{~}5% methanol and ^{~}5% isopropanol) was added to the reaction mixture at a constant flow rate over 1.5 hours. After the reaction mixture was held at 70° C. for a total of 20 hours, the particles were separated from the reaction slurry by filtration. The particles were then washed with methanol, followed by tetrahydrofuran (THF), and followed by acetone. The final product was dried in vacuum oven at 45° C. overnight. 91.8 g of monodisperse 2.3 m polymer particles were obtained.

Other methods for forming particles are known in the art (see, e.g., U.S. Patent Publication Nos 2019/0322783 and 2024/0362428, the methods of forming particles of which are incorporated by reference herein).

In some embodiments, a hydrophilic surface or layer may be formed on the outer surface of the particle core. An exemplary method of forming a hydrophilic surface on a particle is as follows. A hydrophilic primer coating solution containing 36.2 g of glycidyl methacrylate (GMA), 7.44 g of ethylene glycol dimethacrylate (EDMA), 8.21 g of polyvinylpyrrolidone (PVP360, average molecular weight 360,000) and 489.4 g of reagent alcohol (90% ethanol, ^{~}5% methanol and ^{~}5% isopropanol). This solution is added into to a mixture containing the nonporous polymer cores at a constant flow rate over about 1.5 hours to form a hydrophilic surface.

In some embodiments, a linker may be bound to the surface of the particle (e.g., the hydrophilic surface of the particle). In some embodiments, the linker is a bifunctional molecule with a first moiety on a first end and a second moiety on a second end. The first moiety is configured to bind to the hydrophilic surface of particle and the second moiety is configured to bind to streptavidin. In some embodiments, the reaction binding the linker to the surface of the particle is the ring opening of an epoxide. Alternative reactions bonding the linker and the particle surface include amide bond formation, cyanogen bromide reaction, or an aldehyde condensation.

In embodiments wherein an epoxy linker is used, the epoxy linker may have the formula of formula (I)

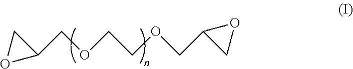

wherein n (the number of ethylene oxide repeating units) is an integer from 1 to 150. In some embodiments, n is 1, 2, 3, 4, 5, 6, 7, 8, 9, 10, 11, or 12. In some embodiments, n is 1, 4, or 9. In some embodiments, n is 1.

### Methods of Forming Streptavidin-Conjugated Particles

After formation of the particle (optionally with a hydrophilic surface, and/or optionally with a linker molecule on the surface of the particle), the particle may be conjugated to streptavidin. For example, in embodiments wherein the particle has an epoxide linker on the surface of the particle, streptavidin may react with the epoxide linker via the ring opening of a surface epoxide. An exemplary method for coupling streptavidin to a particle is described in Example 2.

### Methods of Forming Biotin Endcapped Streptavidin-Conjugated Particles

In some embodiments, the streptavidin-conjugated particles may be packed in a chromatography column (e.g., an affinity chromatography column) which is then connected to a liquid chromatography device. The chromatography column may be prepared as follows. The nonporous particles functionalized with streptavidin are packed into a chromatography column, resulting in a streptavidin column. A number of column sizes and materials are suitable for use in the methods disclosed herein. In some embodiments, the column material is stainless steel, polyetheretherketone (PEEK) lined steel, titanium, or a stainless steel alloy such as MP35n. In some embodiments, the column has an internal diameter ranging from 75 μm to 4.6 mm. In some embodiments, the column has a length between 5 to 300 mm. In a preferred embodiment, the column has an internal diameter between 1 to 2 mm and a length between 5 to 20 mm. The column surface can be unmodified or modified to generate a high-performance surface. Chromatography columns suitable for use with the methods disclosed herein are compatible with any standard liquid chromatography system, including high-performance liquid chromatography (HPLC) systems, ultra-high performance liquid chromatography (UHPLC) systems, and fast protein liquid chromatography (FPLC) systems. The pump system used to pump fluids across the plurality of particles in the chromatographic devices include UHPLC system pumps, HPLC system pumps, and FPLC system pumps. These pump-column systems can be connected to a post-column detector (UV, TUV, PDA, RI, MALS, MS, FL) or they can flow without attachment to a detector. Multiple columns can be coupled in series or in parallel using tubing to increase throughput. The effluent of the columns can be isolated and reused or directed to suitable waste container.

In some embodiments, the liquid chromatography system is connected in series to a detector. Detectors suitable for use in the methods disclosed herein include detectors for ultraviolet spectroscopy, fluorescence spectroscopy, and/or mass spectrometry. In some embodiments, the liquid chromatography system is connected in series to a detector for ultraviolet spectroscopy. In some embodiments, the liquid chromatography system is connected in series to a detector for fluorescence spectroscopy. In some embodiments, the liquid chromatography system is connected in series to detector for mass spectrometry. In some embodiments, the liquid chromatography system is connected to one or more of the detectors in series.

The liquid chromatography device is configured to flow a solution over the packed column of streptavidin-conjugated particles. In some embodiments, the solution includes both a biotinylated affinity agent and free biotin. In some embodiments, streptavidin-conjugated particles are prepared in the chromatography column and the solution including both a biotinylated affinity agent and free biotin and flown through the liquid chromatography device, thereby biotinylating the streptavidin-conjugated particles.

An advantage of the method of pumping the solution including the biotinylated affinity agent and biotin across a bed of particles packed into a device includes precise metering of reagents, contact times and ability to use post column detectors (e.g., use of detector to monitor amount of biotinylated molecule eluting from column versus loading on the column).

After flowing the solution through the plurality of particles packed in the column, the chromatographic device can be washed with water, PBS buffer or storage buffer, and then stoppered or enclosed to prevent evaporation and, if desired, stored in a refrigerator until ready for use.

In some embodiments, a column packed with a plurality of functionalized streptavidin particles can be washed with water, a buffer or storage solution, and/or an acetonitrile-based solution (e.g., 20% acetonitrile and 1% phosphoric acid) prior to adding the solution containing biotinylated affinity agent. The column packed with the plurality of functionalized streptavidin particles can be stored prior to the loading of the biotinylated affinity agent.

### Methods of Determining Streptavidin Leachate

Streptavidin leachate can be monitored by UV absorbance, for example at 280 nm. For example, particles, monoliths, or membranes including biotin endcapped streptavidin-conjugated particles may be washed with a mobile phase and the eluent monitored by UV at 280 nm. As the biotin endcapped streptavidin of the particles, monoliths, or membranes described herein reduce streptavidin leachate, little to no absorbance following washing with the mobile phase will be detected.

Streptavidin leachate can be tested by flowing a mobile phase across the column including a plurality of streptavidin-conjugated particles, a streptavidin-conjugated monolith, or a biotin endcapped streptavidin-conjugated membrane. Eluent from the column, monolith, or membrane may be monitored by UV absorbance at 280 nm for the presence of streptavidin monomers in the eluent. Eluent may further be detected using a mass spectrometer to detect the presence of streptavidin monomers as determined by molecular weight.

The mobile phase may include a buffer, such as phosphate buffered saline (PBS). The mobile phase may further include an organic solvent such as, but not limited to, acetonitrile, methanol, or isopropanol. The mobile phase may further include an acid, such as phosphoric acid. Additionally or alternatively, the mobile phase may include a detergent. The concentration of the organic solvent, detergent, and/or acid may be adjusted as would be understood by one of ordinary skill in the art.

In some embodiments, the biotin endcapped streptavidin-conjugated particles, biotin endcapped streptavidin-conjugated monolith, or biotin endcapped streptavidin-conjugated membrane results in no detectable streptavidin leachate as determined by mass spectrometry.

Accordingly, the biotin endcapped streptavidin-conjugated particles, monoliths, or membranes described herein may result in reduced streptavidin leachate as measured by UV absorbance. In some embodiments, the UV absorbance following washing with a mobile phase is less than 10 mAU.

Alternatively, reduction in streptavidin leachate may be measured as a reduction in peak area following washing with a mobile phase. For said measurement, a comparison is made between a particle, membrane, or monolith having biotin endcapped streptavidin versus a particle, membrane, or monolith having streptavidin which includes only a biotinylated affinity agent and does not include free biotin. In some embodiments, the 4th peak is used as the measurement for determining reduction in streptavidin leachate. In some embodiments, the biotin endcapped streptavidin-conjugated particle, membrane, or monolith results in an 85% reduction in absorbance as measured at the 4th peak. In some embodiments, the streptavidin-conjugated particle, membrane, or monolith results in a 90% or 95% reduction in absorbance as measured at the 4th peak.

### Systems of Biotin Endcapped Streptavidin-Conjugated Particles, Monoliths, or Membranes

The biotin endcapped streptavidin-conjugated particles, monoliths, or membranes may be used to prepare an affinity chromatographic column, an affinity monolith, or an affinity membrane, respectively. In some embodiments, a liquid chromatography device which is used to endcap a streptavidin-conjugated particle may be further utilized in a chromatography experiment (e.g., without removal of the biotin endcapped streptavidin-conjugated particle).

A number of column sizes and materials are suitable for use. In some embodiments, the column material is stainless steel, polyetheretherketone (PEEK) lined steel, titanium, or a stainless steel alloy such as MP35n. In some embodiments, the column material is plastic. In some embodiments, the column has an internal diameter ranging from 75 μm to 4.6 mm. In some embodiments, the column has a length between 5 to 300 mm. The column surface can be unmodified or modified to generate a high-performance surface. Chromatography columns suitable for use with the methods disclosed herein are compatible with any standard liquid chromatography system, including high-performance liquid chromatography (HPLC) systems, ultra-high performance liquid chromatography (UHPLC) systems, and fast protein liquid chromatography (FPLC) systems.

In some embodiments, the liquid chromatography system is connected in series to a detector. Detectors suitable for use in the methods disclosed herein include detectors for ultraviolet spectroscopy, fluorescence spectroscopy, and/or mass spectrometry. In some embodiments, the liquid chromatography system is connected in series to a detector for ultraviolet spectroscopy. In some embodiments, the liquid chromatography system is connected in series to a detector for fluorescence spectroscopy. In some embodiments, the liquid chromatography system is connected in series to detector for mass spectrometry. In some embodiments, the liquid chromatography system is connected to one or more of the detectors in series.

In some embodiments, the interior surfaces of the column are treated to reduce non-specific binding and enhance overall efficiency of the liquid chromatography system. In particular, an alkylsilyl coating or other high-performance surface is provided to limit or reduce non-specific binding of a sample with walls or interior surfaces of a column body. Without wishing to be bound by theory, it is believed that an alkylsilyl coating covering metal surfaces prevent or minimize contact between fluids passing through the column body and the interior surfaces of the column. Typically, the alkylsilyl coating is applied to metal surfaces defining what is known as a wetted path of the column. A metal wetted path includes all surfaces formed from metal that are exposed to fluids during operation of the chromatographic column. The metal wetted path includes not only column body walls, but also metal frits disposed within the column.

In general, the alkylsilyl coating is applied through a vapor deposition technique. Precursors are charged into a reactor in which the part to be coated is located. Vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating is applied to other portions of the liquid chromatography system. For example, the alkylsilyl coating can be applied to metal components residing upstream and downstream of the column. Specifically, the alkylsilyl coating can be applied to an injector of the liquid chromatography system and to post column tubing and connectors.

In one embodiment, the alkylsilyl coating includes a hydrophilic, non-ionic layer of polyethylene glycol silane. In another embodiment, the alkylsilyl coating is formed from one or more of the following precursor materials bis(trichlorosilyl)ethane or bis(trimethoxysilyl)ethane. Other embodiments of alkylsilyl coatings suitable for use with the present technology are described in U.S. Patent Publication No. 2019/0086371 and U.S. Application Publication No. 2022/0118443.

### EXAMPLES

The following examples are meant to illustrate the invention and are not meant to limit the invention in any way.

### Example 1: Addition of an Epoxy Linker to Hydrophilic Particles

The nonporous, epoxy-modified hydrophilic particles for use in the disclosed methods were prepared as follows. As a first step, 1500 g of reagent alcohol (90% ethanol, ~5% methanol, and ~5% isopropanol), 45.1 g of polyvinylpyrrolidone (PVP-40), 4.8 g of 2,2′-Azobis(2-methylpropionitrile), 5.9 g of Triton™ N-57, and 81.7 g of styrene were charged into a reactor. After the reactor was purged with nitrogen gas, the reaction mixture was heated to and maintained at 70° C. with stirring for 3 hours.

After three hours, a solution containing 110.4 g of divinylbenzene (80% by weight), 39.7 g of PVP-40, 510 g of reagent alcohol, and 100.2 g of p-xylene was added to the reaction mixture at a constant flow rate over two hours. Following this step, a primer coating solution containing 26.0 g of glycidyl methacrylate (GMA), 26.0 g of ethylene glycol dimethacrylate (EDMA), 36.4 g of PVP-40, and 560 g of reagent alcohol were added to the reaction mixture at a constant flow rate over 1.5 hours.

The reaction mixture was maintained at 70° C. for a total of 20 hours, after which the particles were separated from the reaction slurry by filtration. The particles were then washed sequentially with methanol, tetrahydrofuran (THF), and acetone. The final product was dried in a vacuum oven at 45° C., resulting in monodisperse 3.5 μm polymer particles. These particles contain a gradient polystyrene/divinylbenzene core with a poly(GMA/EDMA) primer. While the above reaction conditions generate 3.5 μm polymer particles, it is understood that particles ranging in sizes from 1.5 μm to 8 μm are within the scope of the disclosure. By altering the concentrations of PVP-40, 2′2-Azobis(2-methylpropionitrile), and Triton N-57, one of ordinary skill in the art could generate a range of particle sizes.

The resultant 3.5 μm, polystyrene/divinylbenzene particles with the poly(GMA/EDMA) primer were then coated with a hydrophilic layer. 70 g of the particles were hydrolyzed in 0.5M H2SO4 at 60° C. for 1-20 hours. The hydrolyzed particles were washed sequentially with MilliQ water and methanol, and then dried under vacuum at 45° C. overnight. The dried particles were added into a 1 L three-necked round bottom flask with an overhead stirring motor, stirring shaft, and stir blade, a water-cooled condenser, a nitrogen inlet, and a probe-controlled heating mantle. 700 mL of anhydrous diglyme (diethylene glycol dimethyl ether) was added, the flask sealed and purged with nitrogen for 15 minutes with moderate stirring. 2.0 g of potassium tert-butoxide was added, and the reaction was raised to 70° C. To generate the hydrophilic layer, a mixture of 10.5 g glycidol, 2.6 g of glyceroltriglycidyl ether, and 14.9 g of anhydrous diglyme was prepared separately and added to the particle mixture in four equal aliquots in 30-minute intervals. The reaction was held at 70° C. for 20 hours, cooled to RT, and filtered. The resulting particles were washed sequentially with water 6 times, methanol 3 times, and then dried under vacuum overnight at 45° C. The following procedure results in a hydrophilic layer that is 2-4% (by weight) of the entire particle.

20 g of the resultant 3.5 μm particles with the hydrophilic coating were added to a mixture of 100 g of ethylene glycol diglycidyl ether (EGDGE) and 100 g of MeOH at room temperature. 1 mL of 50% sodium hydroxide in water was added and the reaction was stirred continuously for 20 h. The particles were isolated by filtration, washed with 40 mL of MeOH ten times, and partially dried under nitrogen flow. The particles were stored for later use in a methanol wet bed at 4° C. The resultant particles have sufficient epoxide content to enable functionalization of the particle surface.

### Example 2: Preparation of a Streptavidin Column

Particles were prepared as described in Example 1 and functionalized with streptavidin. 1.5 g of particles were mixed in 7 mL of a 50-100 mM buffer (pH 8-9.2). To this, 1.5 mL of a 10 mg/mL solution of streptavidin (15 mg) was added. Next, 21.4 mL of a buffer containing a salting out agent was added dropwise. The reaction was then stirred for 20 hours between 24-37° C. The buffer system, salting out agent and its concentration together I temperature of the reaction can be adjusted to manipulate the extent of streptavidin coverage on a given particle, as shown in Table 1.

Following the 20 h incubation, lg of ethanolamine in 4 mL of a buffer solution (e.g., sodium phosphate) was added and the reaction stirred at RT for 3 hours. Particles were then isolated by filtration and washed. The washing process includes: step 1: three times pH4 water (i.e., adjusted with HCl); step 2: three times with water or water/organic solvent mixture as described in Table 1; step 3: three times with water; and step 4: twice with storage buffer (100 mM PBS, PH 7.3, 0.0200 sodium azide). The particles were stored in a sealed container as a slurry in storage buffer (~10 mL buffer/g of particle) at 4° C. Streptavidin coverage of the particles was determined using a standard bicinchoninic acid assay (BCA). Maximum binding capacity was estimated using the ratio of the molecular weight of streptavidin and a biotinylated antibody multiplied by the binding valency of streptavidin (4) as shown in Table 1.

TABLE 1
Particles Functionalized With Streptavidin

| | | | | | | Maximum |
|---|---|---|---|---|---|---|
| | | | | | Streptavidin | Binding |
| | | | | Particle | Coverage | Capacity |
| Product | Buffer | Salting Out | | Wash 2^{nd} | (μg/mg | (μg IgG/mg |
| # | System | Agent | Temp | Step | particle) | particle) |
| 2a | Phosphate | 1.7M | 24° C. | Water | 2.2 | 24 |
| | Buffer | Ammonium | | | | |
| | (pH8) | Sulfate | | | | |
| 2b | Phosphate | 2.85M | 24° C. | Water | 3.3 | 36 |
| | Buffer | Ammonium | | | | |
| | (pH8) | Sulfate | | | | |
| 2c | Phosphate | 2.85M | 37° C. | Water | 4.6 | 50 |
| | Buffer | Ammonium | | | | |
| | (pH8) | Sulfate | | | | |
| 2d | Phosphate | 2.85M | 37° C. | Acetonitrile/ | 5.2 | 57 |
| | Buffer | Ammonium | | Water | | |
| | (pH8) | Sulfate | | (1/3) | | |
| 2e | Phosphate | 2.85M | 37° C. | Dimethyl | 4.6 | 50 |
| | Buffer | Ammonium | | sulfoxide/ | | |
| | (pH8) | Sulfate | | Water (1/9) | | |
| 2f | Carbonate- | 1.5M | 37° C. | Water | 3.9 | 43 |
| | bicarbonate | Sodium | | | | |
| | Buffer | Sulfate | | | | |
| | (pH9.2) | | | | | |
| 2g | Carbonate- | 1.5M | 37° C. | Dimethyl | 4.0 | 44 |
| | bicarbonate | Sodium | | sulfoxide/ | | |
| | Buffer | Sulfate | | Water (1/9) | | |
| | (pH9.2) | | | | | |
| 2h | Carbonate- | 1.5M | 24° C. | Dimethyl | 3.4 | 37 |
| | bicarbonate | Sodium | | sulfoxide/ | | |
| | Buffer | Sulfate | | Water (1/9) | | |
| | (pH9.2) | | | | | |

### Example 3: Coupling of Biotin or a Biotinylated Antibody to Streptavidin

The ability for the column prepared in Example 1 to bind to a solution including biotin and a biotinylated antibody to bind to accessible binding sites of streptavidin was determined.

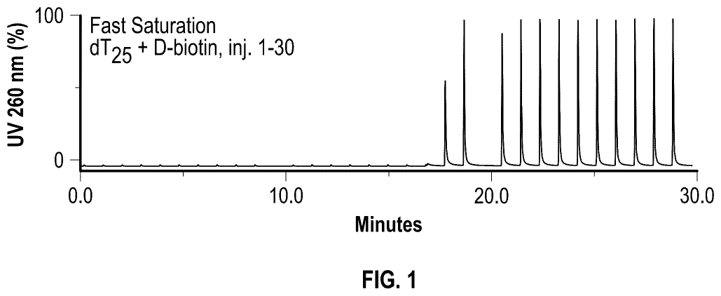

Columns were washed with a mobile phase of 100 mM sodium phosphate (pH 7.4) at a flow rate of 0.15 mL/min. with D-biotin and a biotinylated affinity agent, was injected with 2 μL injections until saturation was observed as measured by UV at 260 nm. Saturation of biotin binding sites is determined by an increase in UV absorbance at 260 nm as, without available streptavidin binding sites, biotin passes through the column and can then be detected in the eluent. As an initial test, biotinylated dT₂₅ was used as a biotinylated affinity agent. The experiment was repeated at different ratios of biotinylated dT₂₅ and free biotin. As shown in FIG. 1, saturation was observed within 30 injections.

### Example 4: Binding Capacity of a Biotin Endcapped Column for mRNA

The binding capacity of biotin endcapped columns prepared as described in Example 2 for mRNA was examined. To demonstrate the fine control over binding capacity of the columns disclosed herein, different ratios of biotinylated affinity agent to biotin were used in column preparation.

To determine column binding capacity, a biotinylated dT₂₅ molecule with a poly A tail was prepared. Four columns were then prepared by first loading a column with streptavidin-conjugated particles, then washing the column with solutions including a mixture of biotinylated dT₂₅ and free biotin. The amount of conjugated biotin was then determined. The first column was washed with 0.114 nmol/mL of biotinylated dT₂₅ with no free biotin, resulting om 28.5 nmol of dT₂₅ conjugated to the particle. The second column was washed with a solution including 0.114 nmol/mL biotinylated dT₂₅ and 0.342 nmol/mL of free biotin (1:3 molar ratio), resulting in 11.4 nmol dT₂₅ conjugated to the particle. The third column was washed with a solution including 0.114 nmol/mL of biotinylated dT₂₅ and 0.9 nmol/mL free biotin (1:8 molar ratio), resulting in 4.56 nmol of dT₂₅ conjugated to the particle. The fourth column was washed with a solution including 0.02565 nmol/mL of biotinylated dT₂₅ and 0.9775 nmol/mL of free biotin (1:40 molar ratio), resulting in 0.97 nmol of dT₂₅ conjugated to the particle.

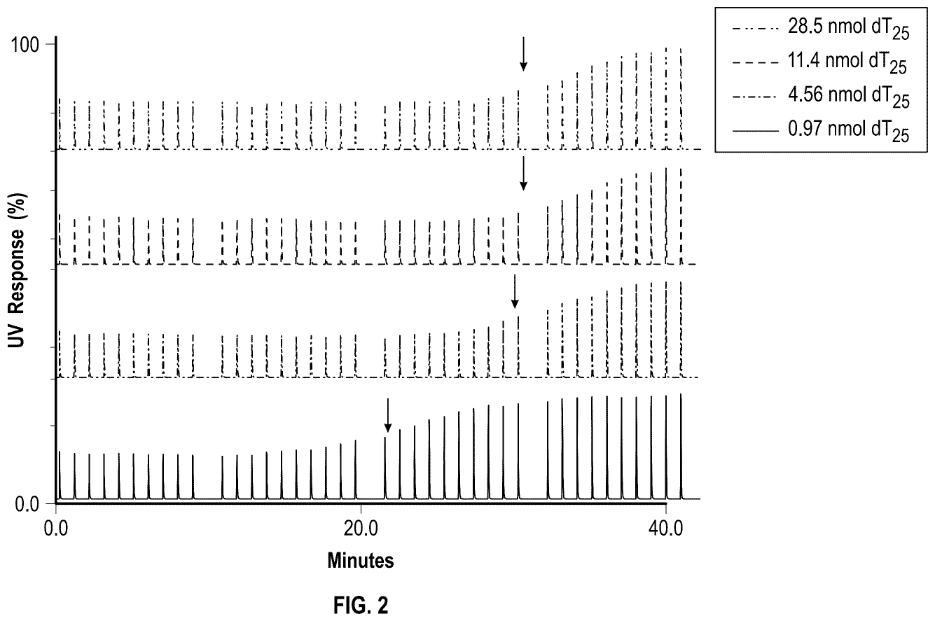

An mRNA sample including a complementary to the biotinylated dT₂₅ was also prepared. Each column was then contacted with a solution including 0.1 M sodium phosphate buffer and 1 mg/mL mRNA at pH 7.5 with a flow rate 0 f 0.15 mL/min. mRNA was injected at 25 C, 2 μL/injection. The results are summarized in Table 2 and FIG. 2.

TABLE 2
mRNA Binding Capacity

| Amount of | Ratio of | mRNA |
|---|---|---|
| Biotinylated dT₂₅ | Biotinylated dT₂₅ to | Coupled |
| (nmol) | Biotin | (pmol) |
| 28.5 | No Biotin | 35.2 |
| 11.4 | 1:3 | 34.5 |
| 4.56 | 1:8 | 32.7 |
| 0.97 | 1:40 | 26.2 |

As shown in Table 2, the mRNA binding capacity of each column remains unaffected up to a biotinylated dT₂₅ to biotin ratio of 1:8, with only a slight reduction in binding capacity noticed up to a ratio of 1:40. This is confirmed by FIG. 2 which shows a consistent UV response to the mRNA up to approximately 30 minutes for the no biotin, 1:3 biotin, and 1:8 biotin samples after which there is an increase in UV response indicating saturation of available mRNA binding sites (the dT₂₅ sites) in the column.

### OTHER EMBODIMENTS

Various modifications and variations of the described invention will be apparent to those skilled in the art without departing from the scope of the invention. Although the invention has been described in connection with specific embodiments, it should not be unduly limited to such specific embodiments. Indeed, various modifications of the described modes for carrying out the invention that are obvious to those skilled in the art are intended to be within the scope of the invention.

Other embodiments are in the claims.

We claim:

1. A method of preparing an affinity chromatographic column, the method comprising contacting a chromatographic column with a solution comprising a mixture of a biotinylated affinity agent and free biotin, wherein:
the chromatographic column comprises a column body formed of a metal or a metal alloy, wherein the column body houses a plurality of streptavidin-conjugated nonporous particles.

2. The method of claim 1, wherein the biotinylated affinity agent and free biotin bind to accessible binding sites of the streptavidin molecules, such that at least 90% of accessible binding sites of the streptavidin molecules are bound with the biotinylated affinity agent or free biotin.

3. The method of claim 2, wherein at least 95% of accessible binding sites of the streptavidin molecules are bound with the biotinylated affinity agent or free biotin.

4. The method of claim 1, wherein the biotinylated affinity agent and free biotin are in a 1:1 molar ratio, 1:3 molar ratio, 1:5 molar ratio, 1:8 molar ratio, 1:10 molar ratio, 1:15 molar ratio, 1:20 molar ratio, 1:25 molar ratio, 1:30 molar ratio, 1:35 molar ratio, 1:40 molar ratio, 1:45 molar ratio, or 1:50 molar ratio.

5. The method of claim 1, wherein the method is performed in less than 20 minutes.

6. The method of claim 1, wherein each particle of the plurality of streptavidin-conjugated nonporous particles comprises:
a nonporous polymer core;
a hydrophilic surface on an outer layer of the nonporous polymer core, wherein one or more streptavidin molecules are conjugated to the hydrophilic surface; and
wherein the average particle size of streptavidin-conjugated nonporous particles is from 1.0 μm to 10 μm.

7. The method of claim 6, wherein the nonporous polymer core has a gradient composition.

8. The method of claim 6, wherein the nonporous polymer core comprises divinylbenzene monomers and polystyrene monomers.

9. The method of claim 6, wherein the hydrophilic surface is selected from the group consisting of: (3-glycidyloxypropyl)trimethoxysilane, (3-glycidyloxypropyl)triethoxysilane, polyacrylate, glycidol, glyceroltriglycidyl ether, and poly(methyl acrylate).

10. The method of claim 6, wherein the one or more streptavidin molecules are conjugated to the hydrophilic surface of the particle via an epoxy linker.

11.-13. (canceled)

14. The method of claim 6, wherein the plurality of streptavidin molecules conjugated to the hydrophilic surface provides a surface coverage of 2-6 μg/mg particle.

15. The method of claim 1, wherein the biotinylated affinity agent is a biotinylated antibody or biotinylated antigen-binding fragment thereof, or a biotinylated oligonucleotide.

16. An affinity chromatographic column generated by the method of claim 1.

17. The affinity chromatographic column of claim 16, wherein at least a portion of an interior surface of the column body is coated with an alkylsilyl material.

18. (canceled)

19. The affinity chromatographic column of claim 17, wherein the alkylsilyl material is a hydrophilic, non-ionic layer of polyethylene glycol silane.

20. The affinity chromatographic column of claim 16, wherein the column is characterized by a reduction in detectable leachate of streptavidin as determined by UV absorbance.

21. The affinity chromatographic column of claim 16, wherein the column is characterized by a leachate absorbance value of <10 mAU as measured by UV absorbance at 280 nm.

22. The affinity chromatographic column of claim 16, wherein the column is characterized by at least an 85% reduction in leachate absorbance as compared to a column that does not comprise streptavidin molecules that are bound with a mixture of the biotinylated affinity agent and the free biotin.

23. (canceled)

24. The affinity chromatographic column of claim 22, wherein the column has at least a 90% reduction or at least a 95% reduction in leachate absorbance.

25. The affinity chromatographic column of claim 16, wherein the column is characterized by no detectable leachate of streptavidin as determined by UV absorbance.
